(19) United States
(12) Patent Application Publication
(10) Pub. No.: US 2026/0249058 A1
(43) Pub. Date: Aug. 27, 2026

(54) TISSUE DILATORS FOR PLEXUS MAPPING

(71) Applicant: Alphatec Spine, Inc., Carlsbad, CA (US)

(72) Inventors: Graham Witherby, Dana Point, CA (US); Thomas Scholl, San Diego, CA (US); Chris Bennett, Escondido, CA (US); Taylor Semingson, San Diego, CA (US)

(21) Appl. No.: 19/060,592

(22) Filed: Feb. 21, 2025

(51) Int. Cl. A61M 29/00

(52) U.S. Cl. CPC A61M 29/00

(57) ABSTRACT

This disclosure relates generally to electrically stimulating dilators for selectively stimulating nerves during surgical procedures. A dilator for use in surgery includes a body extending from a proximal end to a distal end, with a clip attachment area at the proximal end of the body for receiving a stimulating clip. The clip attachment area includes an anterior electrode and a posterior electrode, the anterior electrode and posterior electrode insulated from each other. The dilator also includes a first conductive pathway extending from the anterior electrode to the distal end of the body and a second conductive pathway extending from the posterior electrode to the distal end of the body. An anterior emitter is at a distal end of the first conductive pathway and a posterior emitter is at a distal end of the second conductive pathway, with the posterior emitter disposed opposite the anterior emitter.

### TECHNICAL FIELD

This disclosure relates generally to electrically stimulating dilators for selectively stimulating nerves during dilation and/or surgical procedures.

### SUMMARY

Disclosed are systems, devices, and/or methods of use thereof regarding electrically stimulating dilators for selectively stimulating nerves during dilation and/or surgical procedures. In various aspects, a dilator system for use in surgery includes a dilator body extending from a proximal end to a distal end. A clip attachment area may be disposed near the proximal end of the dilator body, with the clip attachment area being electrically conductive. The dilator system may also include a first emitter patch at the distal end and a first conductive pathway extending from the clip attachment area to the first emitter patch. The first conductive pathway electrically connects the clip attachment area to the first emitter patch. The dilator system may further include a second emitter patch at the distal end, the second emitter patch opposing the first emitter patch, and a second conductive pathway extending from the clip attachment area to the second emitter patch. The second conductive pathway electrically connects the clip attachment area to the second emitter patch. The first and second conductive pathways may be electrically insulated from each other. Additionally, the dilator system may include a clip receivable by the clip attachment area, the clip for conducting an electrical signal to the first emitter patch or the second emitter patch.

In various aspects, a dilator for use in surgery includes a body extending from a proximal end to a distal end. The dilator may also include an attachment area at the proximal end of the body, with the attachment area for receiving a stimulating clip, and the attachment area having an anterior electrode and a posterior electrode. The anterior electrode and posterior electrode may be insulated from each other. The dilator may further include a first conductive pathway extending from the anterior electrode to the distal end of the body and a second conductive pathway extending from the posterior electrode to the distal end of the body. Additionally, the dilator may include an anterior emitter at a distal end of the first conductive pathway and a posterior emitter at a distal end of the second conductive pathway, with the posterior emitter disposed opposite the anterior emitter.

In various aspects, a method of manufacturing a dilator includes selecting a body of the dilator, the body extending from a proximal end to a distal end, and disposing a base insulative layer about the body. The method may also include creating a clip attachment portion having a plurality of electrodes near the proximal end of the body. Further, the method may include creating a first conductive pathway, the first conductive pathway extending from the clip attachment portion to a first emitter patch at the distal end of the body. Still further, the method may include creating a second conductive pathway, the second conductive pathway opposite from the first conductive pathway and extending from the clip attachment portion to a second emitter patch at the distal end of the body. Additionally, the method may include insulating the first conductive pathway from the second conductive pathway.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the Drawings:

FIG. 1A illustrates a perspective view of a dilator according the present disclosure, and FIG. 1B illustrates a close-up view of a proximal end of the dilator;

FIG. 2 illustrates an exploded view of the proximal end of the dilator;

FIG. 3A illustrates a distal end of the dilator and FIG. 3B illustrates an exploded view of the distal end of the dilator;

FIG. 4A illustrates a body of the dilator of FIG. 1A and FIG. 4B illustrates a close-up view of the proximal end of the body;

FIGS. 5A and 5B illustrate close-up views of the proximal end of the body with a base insulative layer;

FIGS. 6A and 6B illustrate close-up views of the proximal end of the body with a first conductive layer;

FIGS. 7A and 7B illustrate close-up views of the proximal end of the body with a second conductive layer;

FIGS. 8A and 8B illustrate close-up views of the proximal end of the body with a first outer insulative layer;

FIGS. 9A and 9B illustrate close-up views of the proximal end of the body with a second outer insulative layer;

FIGS. 10A and 10B illustrate close-up views of the proximal end of the body with first and second conductive bridges;

FIGS. 11A and 11B illustrate close-up views of the proximal end of the body with markings or indicia;

FIGS. 12A and 12B illustrate the dilator having received a clip near the proximal end of the dilator;

FIG. 13 illustrates the clip and accessories for connecting the clip to a neuromonitoring system;

FIG. 14 illustrates the clip;

FIGS. 15A-15C illustrate various embodiments of a dilator according to the present disclosure;

FIG. 16 illustrates one embodiment of an intraoperative monitoring system including the dilator of FIGS. 1 through 12B and the clip of FIGS. 12A through 14; and

FIGS. 17-19 are flowcharts of example methods according to the present disclosure.

### DETAILED DESCRIPTION

Dilators are surgical instruments designed to dilate or expand narrow passages, cavities, and orifices in the body for ease of examinations and procedures. Some dilators provide electrical conductivity to stimulate nerves in the dilated area to ensure proper placement and depth of the dilator and for placement or use of subsequent surgical instruments. These dilators have a clip attachment area near the proximal end that allows for the attachment of a triggered EMG clip. This area is electrically coupled to an emitter patch at the distal end of the dilator, typically using the aluminum base material of the dilator as the conductive pathway. A single emitter patch requires the user to rotate the dilator during use in order to stimulate in the anterior or posterior direction. Unfortunately, rotation of the dilator introduces inadvertent biases to the location of the distal end and the location the emitter patches are actually stimulating. Rotation also introduces user error, as the rotational movements are very small and incremental, so even small deviations can produce large margins of error in stimulation.

Conventional dilators generally include one discrete emitter patch at the distal end of the dilator. To direct the emitter patch in both a posterior and anterior direction requires rotating the dilator, which introduces too much user error into the collected data as such rotations are not exactly 180 degrees to achieve a true anterior/posterior image of the nerves. Another risk of rotating the dilator is inadvertent movement of the distal tip, which can cause damage to surround nerve tissue.

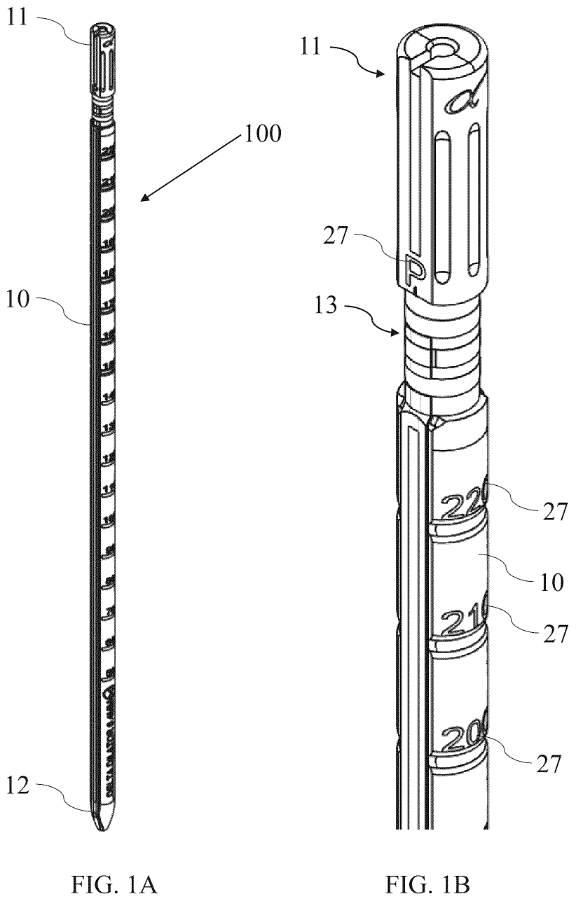

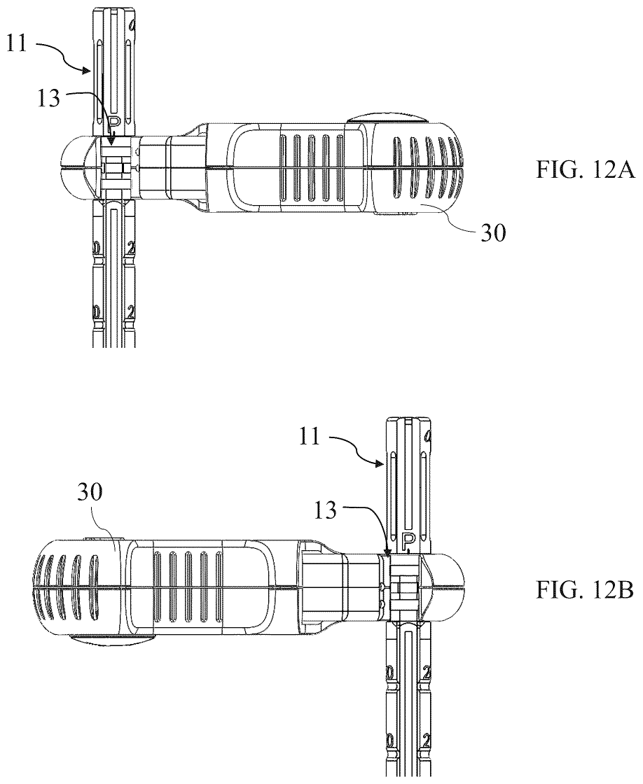

FIGS. 1A through 3B illustrate a dilator 100 according the present disclosure. The dilator 100 includes a body 10 extending between a proximal end 11 and a distal end 12. The body may be formed of a conductive or insulative material, as desired. Included or defined near the proximal end 11 is a clip attachment area 13 (also referred to as an attachment area). The clip attachment area 13 may be electrically conductive and may receive a triggered EMG clip (see the clip 30 of FIGS. 12A through 14) for conducting electrical signals to the distal end 12 of the dilator 100.

The clip may include an actuator to deliver or conduct electrical signals to the distal end 12 of the dilator 100. In some embodiments, the clip is in communication with a neuromonitoring system which selectively conducts electrical signals to the distal end 12 of the dilator 100. Notably, the dilator 100 includes a single clip attachment area 13, thereby simplifying use of the dilator 100. The single clip attachment area 13 can accommodate the clip in any orientation, while still allowing for selective stimulation of anterior or posterior tissues in contact with the dilator 100.

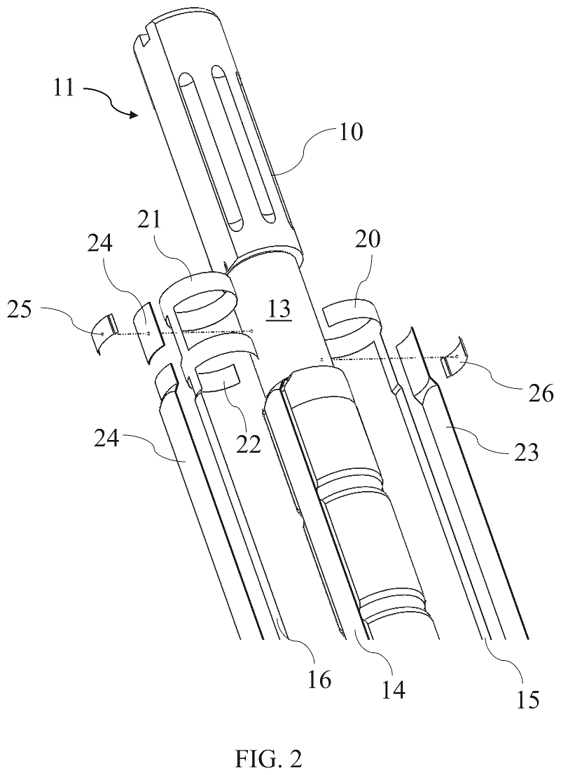

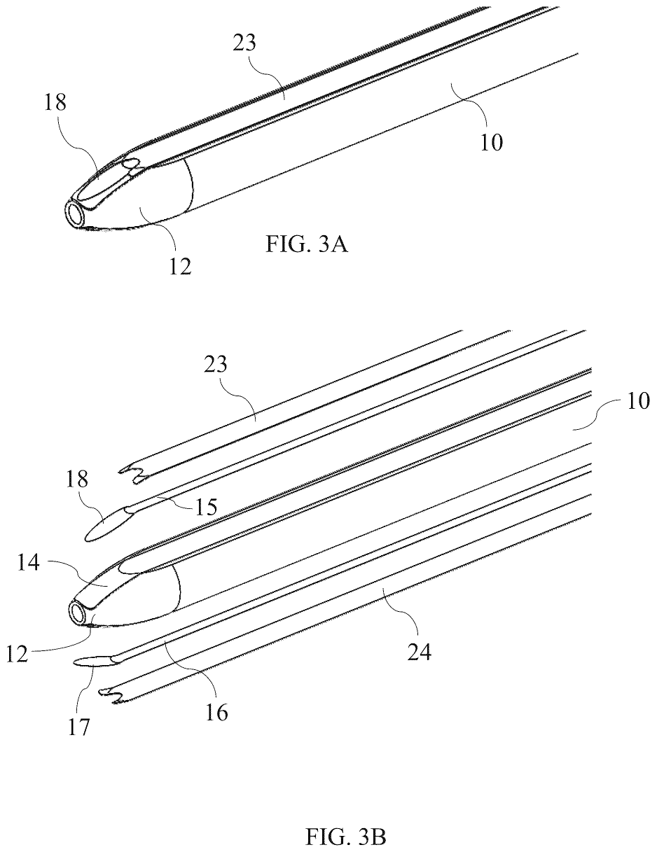

Referring to FIGS. 2 through 3B, the dilator 100 has a plurality of layers, which may include one or more conductive layers and one or more insulative layers. For example, the dilator 100 includes a base or inner insulative layer 14 disposed on a portion the body 10 of the dilator 100. The base insulative layer 14 may extend from the clip attachment area 13, down the body 10, and to the distal end 12 of the dilator 100. The dilator also includes a first conductive pathway 15 and a second conductive pathway 16, both disposed over the base insulative layer 14 and both extending from the clip attachment area 13 to the distal end 12 of the dilator 100. At the clip attachment area 13, a portion of the first conductive pathway 15 may form a first electrode 20. The first electrode 20 may conduct electrical signals to an anterior side of the dilator 100, thereby stimulating anterior tissues. Similarly, a portion of the second conductive pathway 16 may form a second electrode 21 and a third electrode 22. The second and third electrodes 21, 22 may conduct electrical signals to a posterior side of the dilator 100, thereby stimulating posterior tissues.

The first conductive pathway 15 terminates at the distal end 12 in a first emitter patch 17; the second conductive pathway 16 terminates at the distal end 12 in a second emitter patch 18. The first and second emitter patches 17, 18 are exposed at the distal end 12 in order to stimulate nerves or other anatomical structures near the distal end 12 of the dilator 100 when the dilator 100 is positioned within a patient.

The dilator 100 may also include a first outer insulative layer 23 disposed on the first conductive pathway 15 and a second outer insulative layer 24 disposed on the second conductive pathway 16. The first outer insulative layer 23 extends from the clip attachment area 13 to the distal end 12 of the dilator, while leaving the first emitter patch 17 exposed at the distal end 12. Similarly, the second outer insulative layer 24 extends from the clip attachment area 13 to the distal end 12 of the dilator, while leaving the second emitter patch 18 exposed at the distal end 12.

The first and second insulative layers 23, 24 are disposed over the first and second conductive pathways 15, 16 in order to increase accuracy of electrical signals conducted along the first and second conductive pathways 15, 16. Additionally, the dilator 100 includes a first bridge 25 (also referred to herein as “a first conductive bridge 25”) for connection with the first electrode 20 and a second bridge 26 (also referred to herein as “a second conductive bridge 26”) for connection with the third electrode 22. When a clip is attached at the clip attachment area 13, pins of the clip may interact with the first electrode 20, the second electrode 21, the third electrode 22, first bridge 25, and/or the second bridge 26, thereby conducting electrical signals to the distal end 12 of the dilator 100.

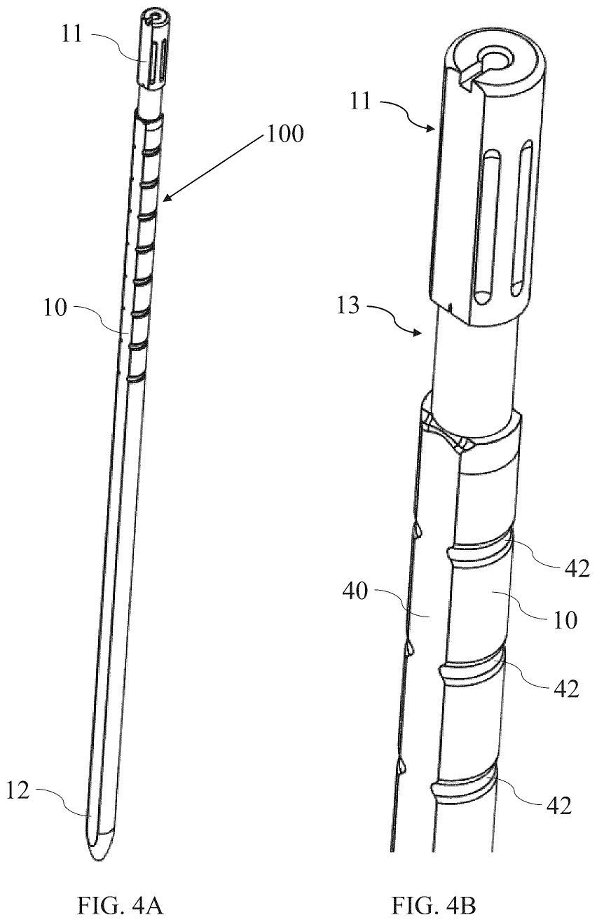

FIGS. 4A through 11B iteratively illustrate the dilator 100 and the various layers disposed about the dilator 100. FIGS. 4A and 4B illustrate the body 10 of the dilator 100, with no layers (conductive or insulative). The body 10 may be formed of insulative or conductive material, as desired. In some embodiments, the body 10 is formed from a metal, such as anodized aluminum. In other embodiments, the body 10 may be formed from a non-conductive metal. Still further, in other embodiments, the body 10 may be formed from a non-conductive material, such as plastic.

The clip attachment area 13 is defined near the proximal end 11. The proximal end 11 may also include a grip or handle portion for a clinician to grasp while inserting the dilator 100 within a patient (or for a tool/handle/etc. to grasp). The body 10 may include planar opposing sides 40 where the layers may be applied or disposed. Alternatively, the body may not have planar sides. The body 10 may include notches or ridges 42 that extend along a portion of a length of the body 10. The ridges 42 may correspond to distance measurements along the length of the body 10 and may include markings or indicia 27 that visually communicate the distance measurements.

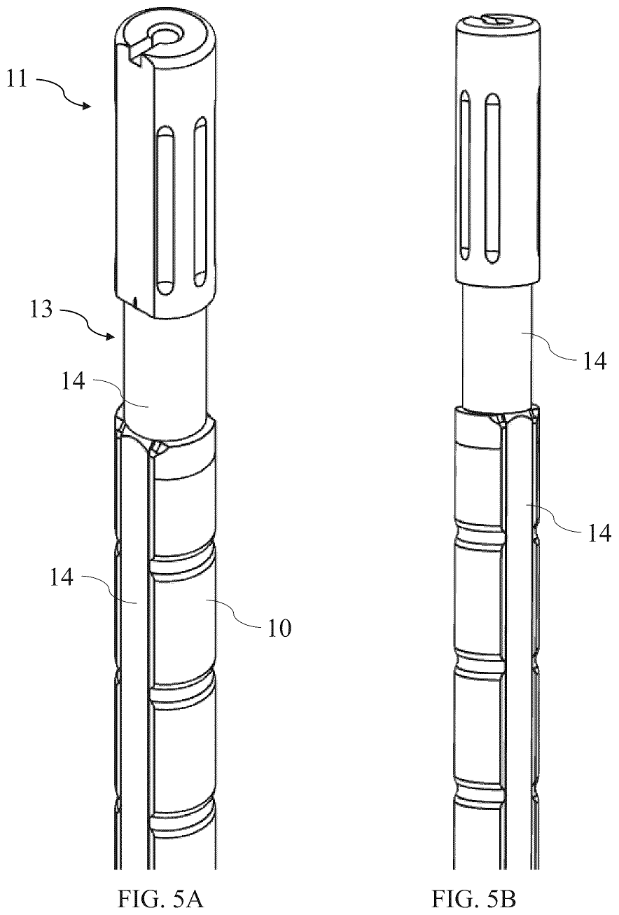

FIGS. 5A and 5B illustrate the body 10 with the base insulative layer 14. When the body 10 is formed from a non-conductive plastic, the base insulative layer 14 may be omitted. When the body 10 is formed from a metal, the base insulative layer 14 is included to insulate the conductive pathways 15, 16 from the underlying conductive material of the body 10. The base insulative layer 14 is disposed about the clip attachment area 13 and extends over at least a portion of the body 10. For example, as illustrated, the base insulative layer 14 is disposed along the planar sides 40 of the body 10. The base insulative layer 14 may be a dielectric material or any appropriate insulative material.

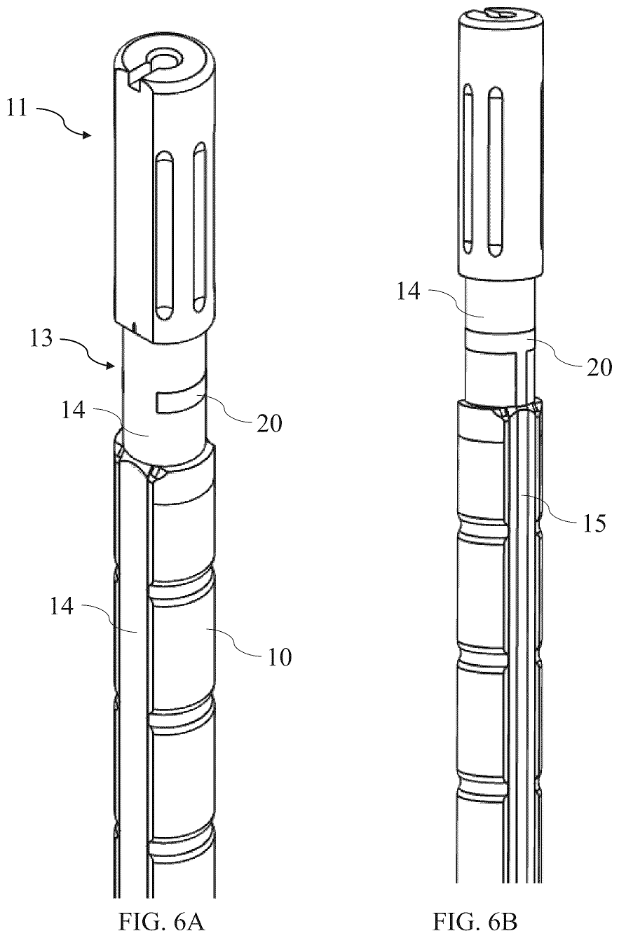

FIGS. 6A and 6B illustrate the body 10 with the base insulative layer 14, the first conductive pathway 15, and the first electrode 20. The first conductive pathway 15 and the first electrode 20 are disposed over the base insulative layer 14 on one side of the body 10. The first electrode 20 may extend partially around a circumference of the clip attachment area 13. The first conductive pathway 15 extends from the first electrode 20, down a planar side 40 of the body 10, and to the distal end 12 of the body 10. Both the first conductive pathway 15 and the first electrode 20 may be formed from a conductive ink, such as a silver conductive ink, or any other suitable conductive material. A conductive ink may be printed onto the dilator 100 over the base insulative layer 14. As the first conductive pathway 15 and the first electrode 20 are disposed over the base insulative layer 14, the first conductive pathway 15 and the first electrode 20 are insulated from the body 10 of the dilator 100.

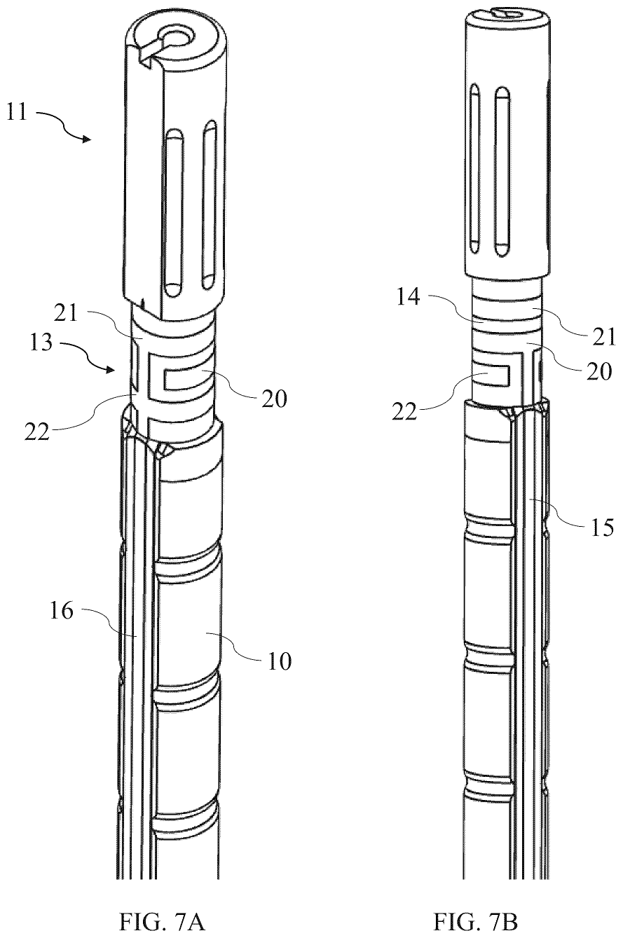

FIGS. 7A and 7B illustrate the body 10 with the base insulative layer 14, the first conductive pathway 15, the first electrode 20, the second conductive pathway 16, the second electrode 21, and the third electrode 22. The second conductive pathway 16, the second electrode 21, and the third electrode 22 are disposed over the base insulative layer 14 on one side of the body 10 opposite the first conductive pathway 15. The second electrode 21 may encircle the clip attachment area 13, such that a portion of the second electrode 21 is positioned proximally relative to the first electrode 20 (see FIG. 7B). The third electrode 22 may extend partially around a circumference of the clip attachment area 13, such that a portion of the third electrode 22 is positioned distally relative to the first electrode 20. The second conductive pathway 16 extends from the second and third electrodes 21, 22, down a planar side 40 of the body 10, and to the distal end 12 of the body 10.

The second conductive pathway 16, the second electrode 21, and the third electrode 22 may also be formed from a conductive ink, such as a silver conductive ink. The conductive ink may be printed onto the dilator 100 over the base insulative layer 14. As the second conductive pathway 16, the second electrode 21, and the third electrode 22 are disposed over the base insulative layer 14, the second conductive pathway 16, the second electrode 21, and the third electrode 22 are insulated from the body 10 of the dilator 100.

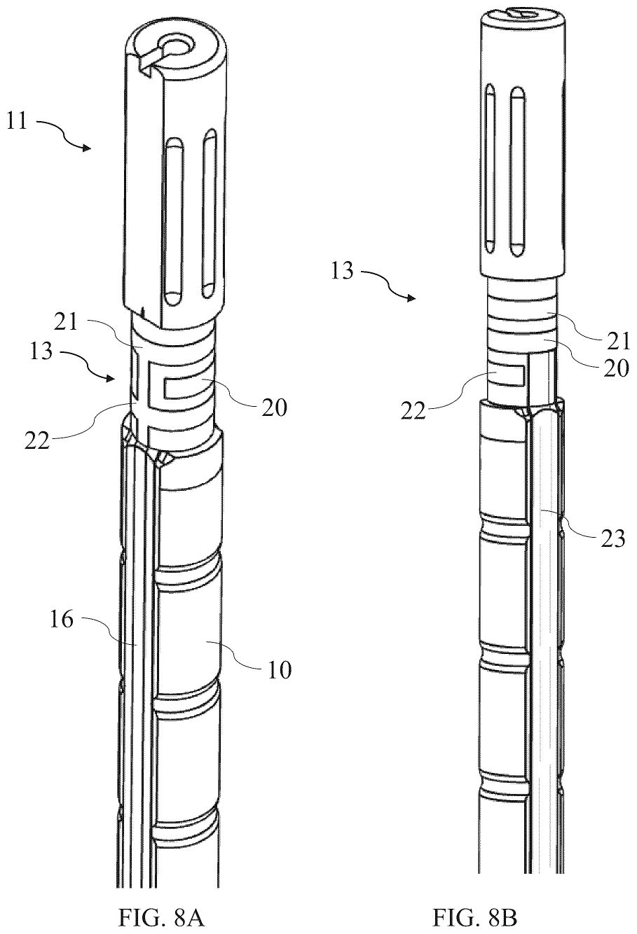

FIGS. 8A and 8B illustrate the body 10 with the base insulative layer 14, the first conductive pathway 15, the first electrode 20, the second conductive pathway 16, the second electrode 21, the third electrode 22, and a first outer insulative layer 23. The first outer insulative layer 23 is disposed along the first conductive pathway 15 and extends from the clip attachment area 13 to the distal end 12 of the body 10. The first outer insulative layer 23 may be substantially continuous from the clip attachment area 13 to the distal end 12. At the distal end 12, the first outer insulative layer 23 terminates before the first emitter patch 17 (FIG. 3B), such that the first emitter patch is exposed for electrical stimulation of tissues adjacent to the first emitter patch 17. The first outer insulative layer 23 also does not cover the first electrode 20, such that the first electrode 20 is exposed for electrical engagement with the clip.

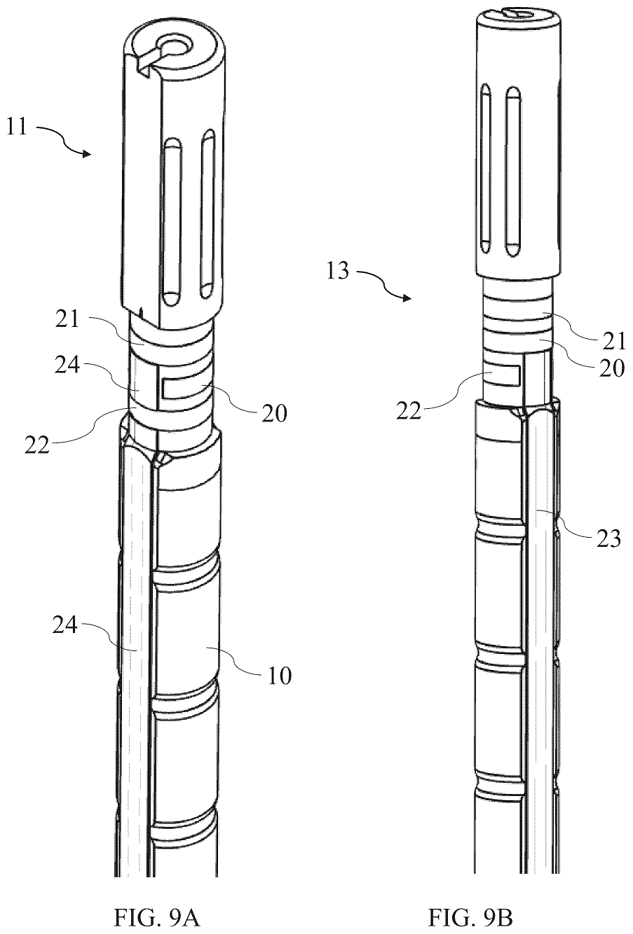

FIGS. 9A and 9B illustrate the body 10 with the base insulative layer 14, the first conductive pathway 15, the first electrode 20, the second conductive pathway 16, the second electrode 21, the third electrode 22, the first outer insulative layer 23, and a second outer insulative layer 24. The second outer insulative layer 24 is disposed along the second conductive pathway 16. A portion of the second outer insulative layer 24 may be disposed at the clip attachment area 13, between the second electrode 21 and the third electrode 22. Each of the second electrode 21 and the third electrode 22 are exposed for electrical engagement with the clip. The second outer insulative layer 24 extends from the clip attachment area 13 to the distal end 12 of the body 10. At the distal end 12, the second outer insulative layer 24 terminates before the second emitter patch 18 (FIG. 3A), such that the second emitter patch is exposed for electrical stimulation of tissues adjacent to the second emitter patch 18.

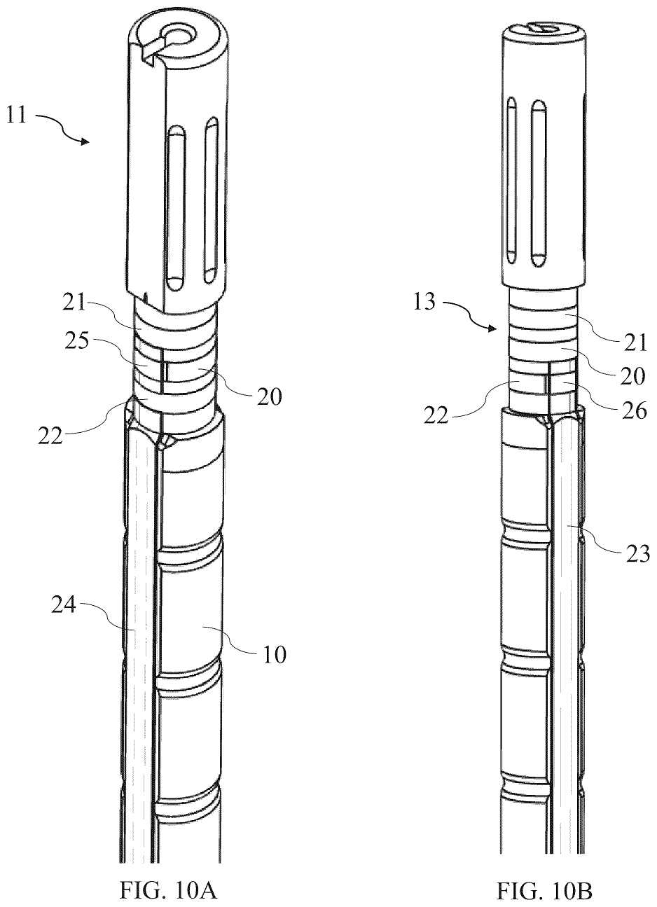

FIGS. 10A and 10B illustrate placement of first and second bridges 25, 26 at the clip attachment area 13. The first and second bridges 25, 26 may be conductive. The first bridge 25 is disposed over a portion of the second outer insulative layer 24. As seen in FIG. 10A, the first bridge 25 overlaps with a portion of the first electrode 20. This overlap places the first bridge 25 in electrical communication with the first conductive pathway 15 through the first electrode 20. When a clip is attached to the clip attachment area 13 from a posterior side of the dilator 100, pins of the clip may come into contact with the first bridge 25. Through the first bridge 25, electrical signals may be conducted to an anterior side of the dilator 100, thereby stimulating anterior tissues. When the clip is attached to the clip attachment area 13 from an anterior side of the dilator 100, pins of the clip may come into contact with the first electrode 20. Through the first electrode 20, electrical signals may be conducted to an anterior side of the dilator 100, thereby stimulating anterior tissues. Thus, no matter what position a clip is attached onto the clip attachment area 13, electrical stimulation will be given to the first electrode.

The second bridge 26 is disposed over a portion of the first outer insulative layer 23. As seen in FIG. 10B, the second bridge 26 overlaps with a portion of the third electrode 22. This overlap places the second bridge 26 in electrical communication with the second conductive pathway 16 through the third electrode 22. When a clip is attached to the clip attachment area 13 from the posterior side of the dilator 100, pins of the clip may come into contact with the third electrode 22. Through the third electrode 22, electrical signals may be conducted to a posterior side of the dilator 100, thereby stimulating posterior tissues. When the clip is attached to the clip attachment area 13 from the anterior side of the dilator 100, pins of the clip may come into contact with the second bridge 26. Through the second bridge 26 and third electrode 22, electrical signals may be conducted to the posterior side of the dilator 100, thereby stimulating posterior tissues.

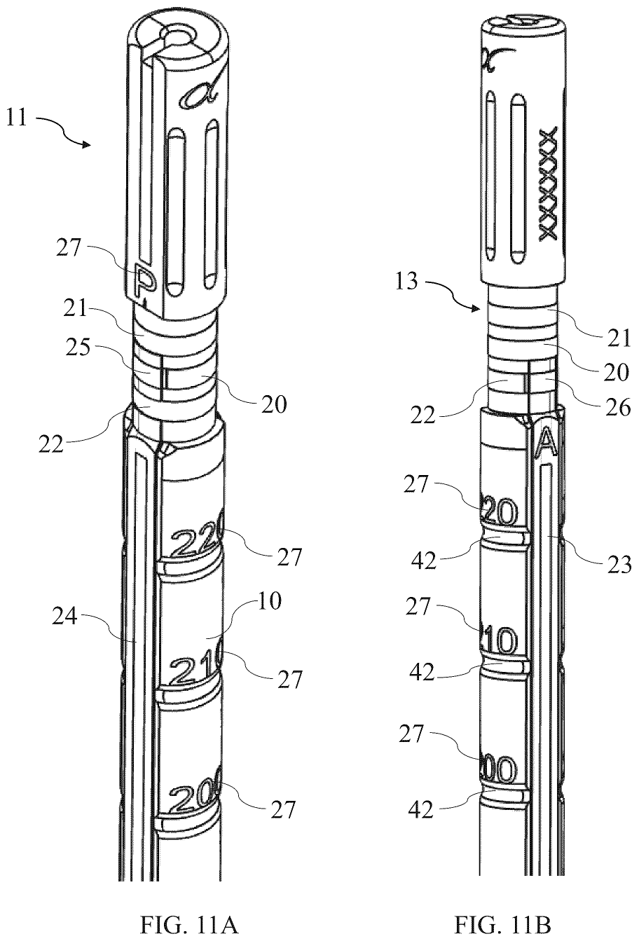

FIGS. 11A and 11B illustrate the dilator 100, with all of the conductive and insulative layers, with optional final markings or indicia 27. The markings 27 may be disposed at the ridges 42 of the body 10 and may correspond to distance measurements (e.g., 200 mm, 210 mm, 220 mm, etc.). The markings 27 may provide a clinician a quick visual guide of how deep the dilator 100 is within a patient. The dilator 100 may also include markings 27 indicating an anterior (A) side of the dilator 100 and a posterior (P) side of the dilator 100. This also provides a quick visual guide to the clinician of what orientation the dilator 100 is placed within the patient.

FIGS. 12A and 12B illustrate the dilator 100 having received a clip 30 near the proximal end 11 of the dilator 100. As shown, the clip 30 is connected or attached to the clip attachment area 13. The clip 30 may be attached in any orientation and still maintain proper electrical contact with the clip attachment area 13. The placement and orientation of the first electrode 20, the second electrode 21, and the third electrode 22 at the clip attachment area 13 ensures that anterior and/or posterior stimulation occurs, independent of clip orientation.

As shown in FIG. 12A, pins of the clip 30 will contact the second electrode 21 and the first electrode 20 in a first orientation. As shown in FIG. 12B, pins of the clip 30 will contact the first electrode 20 and the third electrode 22 in a second orientation. In this illustrated embodiment, the first electrode 20 is always positioned between the second and third electrodes 21, 22, such that a pin of the clip 30 will always contact the first electrode 20. Whether the pins of the clip 30 contact the second electrode 21 or the third electrode 22 depends on the orientation the clip 30 is attached to the clip attachment area 30. In alternative embodiments, a reversed configuration will achieve the same outcome of ensuring proper connection irrespective of how the clip 30 is orientated relative to the dilator 100.

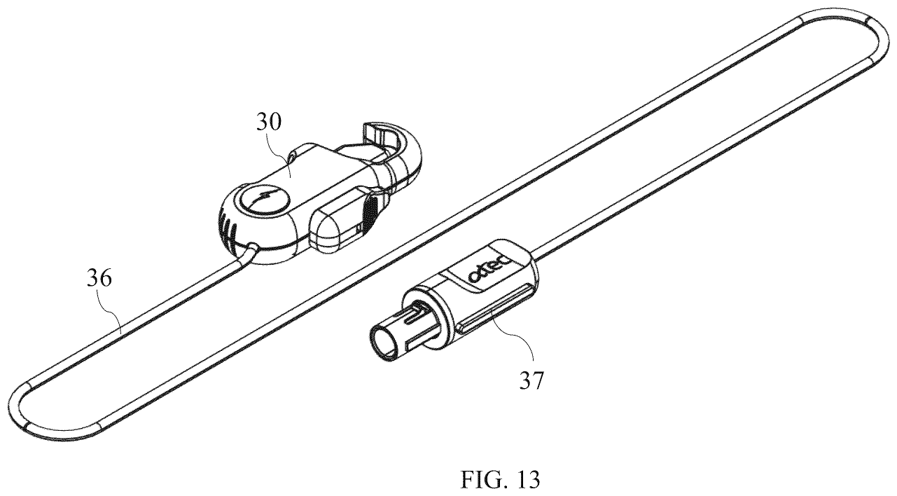

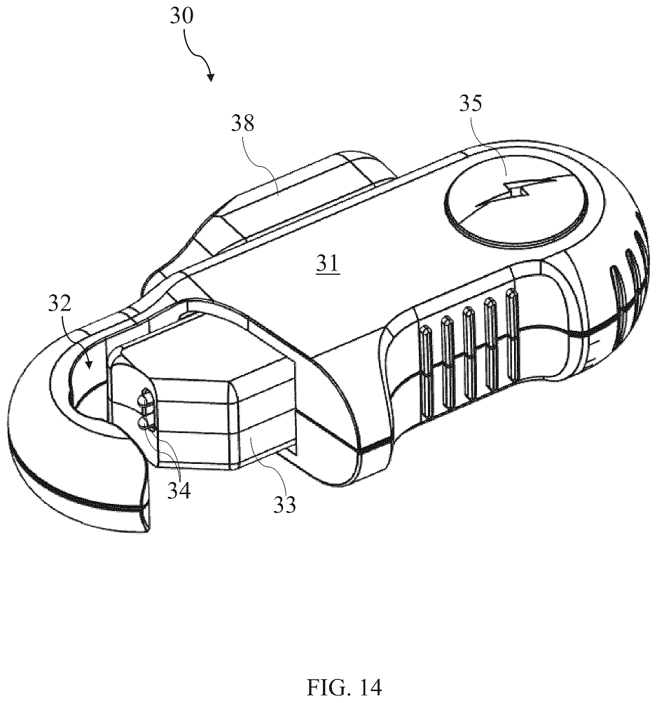

Referring to FIGS. 13 and 14, the clip 30 includes a body 31 and an opening 32 defined by the body 31. The opening 32 is for engaging the clip attachment area 13. The clip 30 also includes a pin body 33, which may be spring-loaded and disengaged by pressing a button 38. The pin body 33 provides a secure mechanism for securing the clip 30 to the clip attachment area 13. The pin body 33 houses pins 34 (two pins 34 are illustrated) for electrical connection with the clip attachment area 13. As described above, one pin 34 will always be in contact with the first electrode 20 for conducting electrical signals to an anterior side of the dilator 100, and the other pin 34 will contact either the second electrode 21 or the third electrode 22 (depending on the orientation of the clip 30) for conducting electrical signals to a posterior side of the dilator 100.

The clip 30 also includes an actuator 35 for triggering the conduction of electrical signals. The actuator 35 may be a button, toggle, switch, slide, etc. or any appropriate actuator 35. The clip 30 may also be in communication with a neuromonitoring system (not illustrated), which may selectively conduct signals for anterior or posterior stimulation upon actuation of the actuator 35. That is, upon actuation of the actuator 35, the neuromonitoring system will determine whether the electrical signal is conducted through the first electrode 20 and first conductive pathway 15 (for anterior stimulation) or through the second or third electrodes 21, 22 and the second conductive pathway 16 (for posterior stimulation). The clip 30 may be connected to the neuromonitoring system through a hardware attachment 37 (e.g., for plugging into a computer or other system), which is connected to the clip 30 via cable 36.

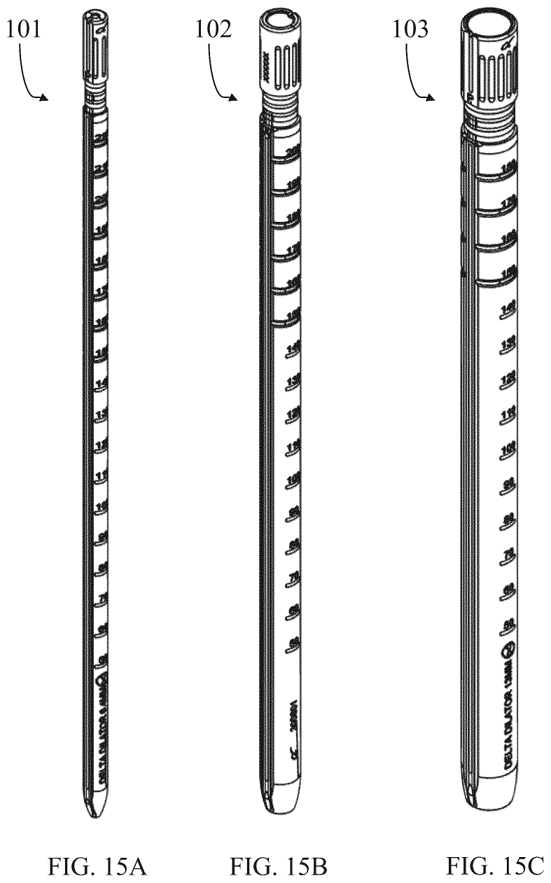

FIGS. 15A-15C illustrate various embodiments of a dilator according to the present disclosure. A first dilator 101 may have the smallest diameter and be used as the initial dilator for expansion. A second dilator 102 may have an intermediate diameter and may be used after the first dilator 101 for increasing expansion. A third dilator 103 may have the largest diameter and may be used as the final dilator for expansion. Each of the first dilator 101, the second dilator 102, and the third dilator 103 may have the structure and layers of the dilator 100 of FIGS. 1 through 12B. Dilators having smaller or larger diameters can also be provided as desired by a clinician to suit a particular patient's needs.

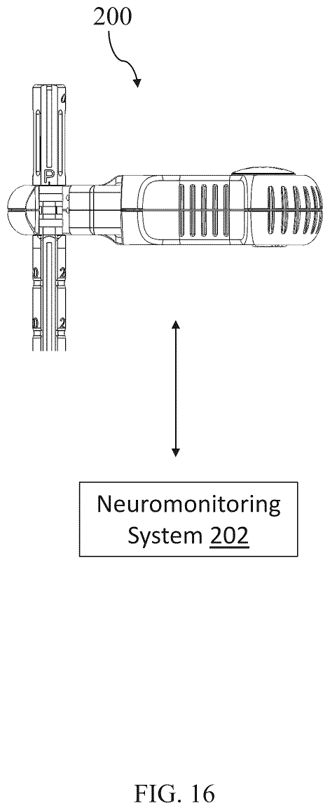

FIG. 16 illustrates one embodiment of an intraoperative monitoring system 200 including the dilator 100 of FIGS. 1 through 12B and the clip 30 of FIGS. 12A through 14. As described, the clip 30 may be in communication with the neuromonitoring system 202. The neuromonitoring system 202 may selectively deliver electrical signals through the dilator 100 for anterior or posterior stimulation.

Although not illustrated, an alternative embodiment of a dilator utilizes two discreet emitter patches as with dilator 100 but in conjunction with a clip that is not configured for simultaneous electrical connection to each emitter patch. For example, the clip has only one pin for transmitting an electrical pulse from a neuromonitoring system down the dilator to one emitter patch at a time. Transmitting to both emitter patches requires a user to move the clip up and down along the dilator to alternatingly contact respective electrodes located at a clip attachment area of the dilator. In some such embodiments, only two electrodes are needed, one each in electrical communication with a respective emitter patch. To utilize such embodiments, a user attaches the clip to one electrode, activates the neuromonitoring system to gather information on nerves located anteriorly (or posteriorly) to the dilator, moves the clip to the other electrode, activates the neuromonitoring system to gather information on nerves located posteriorly (or anteriorly) to the dilator, and repeats this process as many times as desired. The dilator is not rotated, but the clip is simply moved up and down the dilator.

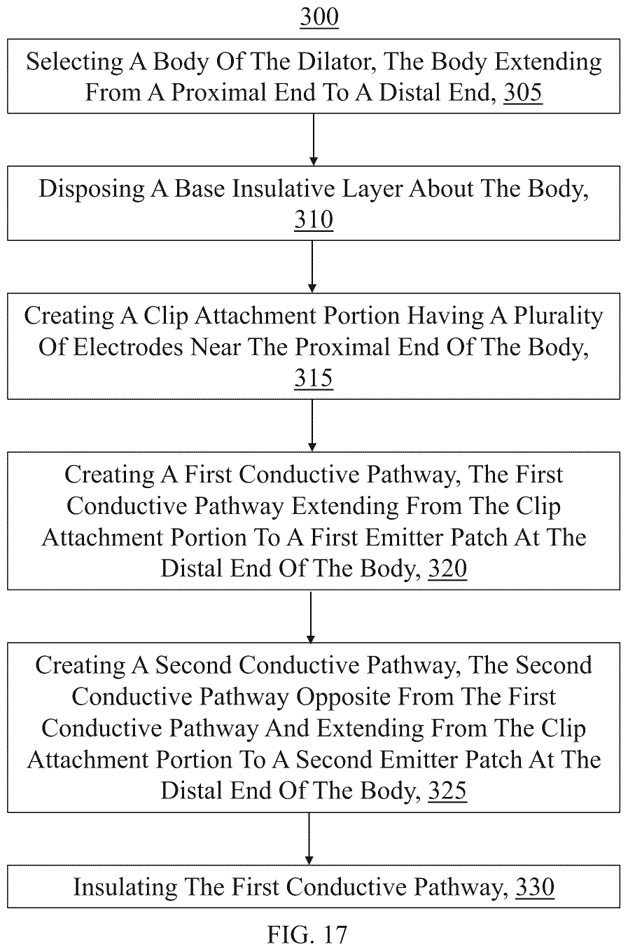

FIGS. 17-19 are flowcharts of example methods according to the present disclosure. FIG. 17 is a flowchart of a method 300 for manufacturing a dilator, such as the dilator 100 described with respect to FIGS. 1 through 12B. The method 300 may include selecting a body of the dilator, the body extending from a proximal end to a distal end, at 305. The method 300 may also include the disposing a base insulative layer about the body, at 310 (this may be optional if the body 10 is formed of insulative material), and creating a clip attachment portion having a plurality of electrodes near the proximal end of the body, at 315. Further, the method 300 may include creating a first conductive pathway, the first conductive pathway extending from the clip attachment portion to a first emitter patch at the distal end of the body, at 320. Still further, the method 300 may include creating a second conductive pathway, the second conductive pathway opposite the first conductive pathway and extending from the clip attachment portion to a second emitter patch at the distal end of the body, at 325. The method 300 may include insulating the first conductive pathway, at 330.

Creating the clip attachment portion having the plurality of electrodes near the proximal end may include applying a first conductive layer over the base insulative layer at the clip attachment portion. The first conductive layer may extend partially around a circumference of the clip attachment portion. Additionally, creating the clip attachment portion may include applying a second conductive layer over the base insulative layer at the clip attachment portion, with the second conductive layer including a top portion and a bottom portion, the top and bottom portions flanking the first conductive layer. Creating the clip attachment portion may additionally include insulating a portion of the first conductive layer and insulating a portion of the second conductive layer. The first electrode may be formed by applying a first conductive patch on an insulated portion of the second conductive layer, thereby forming a first electrode, where the first electrode is in electrical communication with the first conductive pathway. The second electrode may be formed by applying a second conductive patch on an insulated portion of the first conductive layer, thereby forming a second electrode, with the second electrode in electrical communication with the second conductive pathway.

Creating the first conductive pathway may include applying a first conductive ink over the base insulative layer at the clip attachment portion to form a first electrode. Additionally, the first conductive pathway is created by extending the first conductive ink from the clip attachment portion to the distal end and forming the first emitter patch at the distal end of the body. Further, the first conductive pathway may be formed by insulating a portion of the first conductive ink and applying a first conductive patch on an insulated portion of the second conductive pathway, the first conductive patch in electrical communication with the first electrode and the first emitter patch. Applying the first conductive ink over the base insulative layer may include printing and/or etching conductive ink onto the base insulative layer. Applying the first conductive patch may include printing the first conductive ink on top of the insulated portion of the second conductive pathway, such that the first conductive ink is electrically insulated from the second conductive pathway, and connecting the first conductive patch to the first electrode.

Creating the second conductive pathway may include applying a second conductive ink over the base insulative layer at the clip attachment portion to form a second electrode. Additionally, the second conductive pathway is created by extending the second conductive ink from the clip attachment portion to the distal end and forming the second emitter patch at the distal end of the body. Further, the second conductive pathway is created by insulating a portion of the second conductive ink and applying a second conductive patch on an insulated portion of the first conductive pathway, the second conductive patch in electrical communication with the second electrode and the second emitter patch. Applying the second conductive ink over the base insulative layer may include printing and/or etching conductive ink onto the base insulative layer.

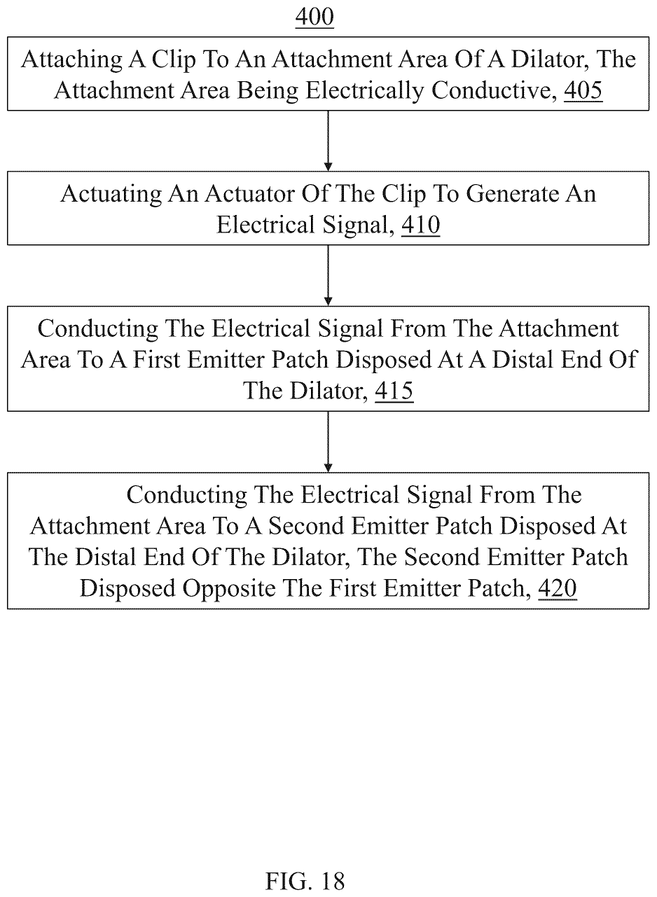

FIG. 18 is a flowchart of a method 400 using a dilator system including a dilator, such as dilator 100, and a clip, such as clip 30. The method 400 may include attaching a clip to an attachment area of a dilator, the attachment area being electrically conductive, at 405. The method 400 may also include actuating an actuator of the clip to generate an electrical signal, at 410, and conducting the electrical signal from the attachment area to a first emitter patch disposed at a distal end of the dilator, at 415. Further, the method 400 may include conducting the electrical signal from the attachment area to a second emitter patch disposed at the distal end of the dilator, the second emitter patch disposed opposite the first emitter patch, at 420. Conducting the electrical signal to the first emitter patch or the second emitter patch does not require rotation of the dilator.

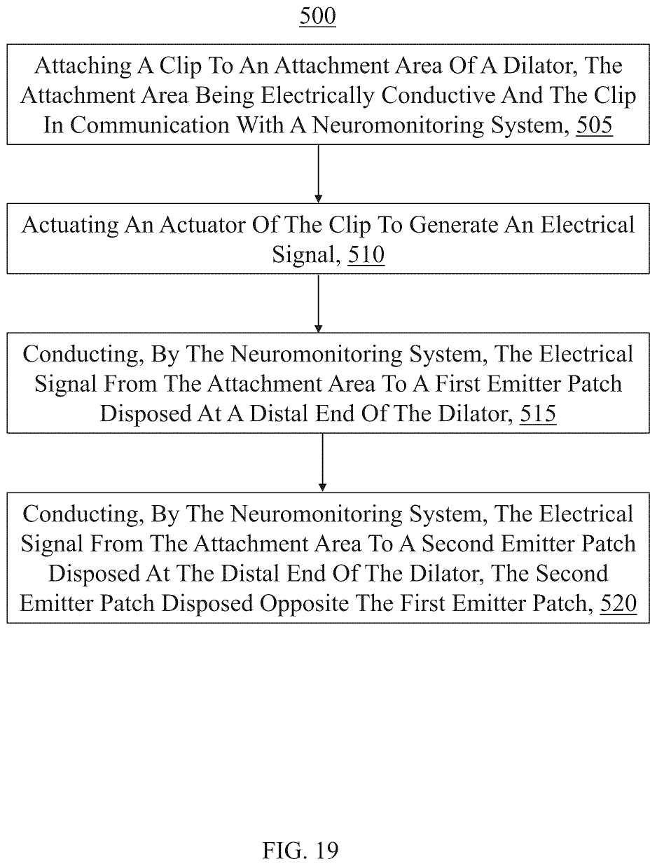

FIG. 19 is a flowchart of a method 500 of using a dilator system, such as the system 200 of FIG. 16. The system may include a dilator 100, such as described with respect to FIGS. 1 through 12B; a clip, such as the clip 30 described with respect to FIGS. 12A through 14; and a neuromonitoring system, such as system 200. The method 500 may include attaching a clip to an attachment area of a dilator, the attachment area being electrically conductive and the clip in communication with a neuromonitoring system, at 505. The method 500 may also include actuating an actuator of the clip to generate an electrical signal, at 510, and conducting, by the neuromonitoring system, the electrical signal from the attachment area to a first emitter patch disposed at a distal end of the dilator, at 515. Further, the method 500 may include conducting, by the neuromonitoring system, the electrical signal from the attachment area to a second emitter patch disposed at the distal end of the dilator, the second emitter patch disposed opposite the first emitter patch, at 520. Conducting the electrical signal to the first emitter patch or the second emitter patch does not require rotation of the dilator.

### Embodiments

Embodiment 1. A dilator system for use in surgery, the dilator system comprising a dilator body extending from a proximal end to a distal end; a clip attachment area near the proximal end of the dilator body, the clip attachment area being electrically conductive; a first emitter patch at the distal end; a first conductive pathway extending from the clip attachment area to the first emitter patch to electrically connect the clip attachment area to the first emitter patch; a second emitter patch at the distal end, the second emitter patch opposing the first emitter patch; a second conductive pathway extending from the clip attachment area to the second emitter patch to electrically connect the clip attachment area to the second emitter patch; and a clip receivable by the clip attachment area, the clip for conducting an electrical signal to the first emitter patch or the second emitter patch.

Embodiment 2. The dilator system of Embodiment 1, wherein the first emitter patch and the first conductive pathway comprise a conductive ink.

Embodiment 3. The dilator system of Embodiment 1 or Embodiment 2, wherein the clip attachment area comprises a first electrode in electrical communication with the first conductive pathway and a second electrode in electrical communication with the second conductive pathway.

Embodiment 4. The dilator system of Embodiment 3, wherein the first electrode and the second electrode are electrically insulated from each other at the clip attachment area.

Embodiment 5. The dilator system of Embodiment 3 or Embodiment 4, wherein the first electrode is disposed distally relative to the second electrode.

Embodiment 6. The dilator system of Embodiment 3 or Embodiment 4, wherein the clip comprises a first pin for alternating electrical connection to the first electrode and the second electrode, which alternating connection is achieved by moving the clip from a first position on the dilator to a second position on the dilator and then back to the first position.

Embodiment 7. The dilator system of Embodiment 3 or Embodiment 4, wherein the clip comprises a first pin for electrical connection to the first electrode and a second pin for electrical connection to the second electrode.

Embodiment 8. The dilator system of Embodiment 3 or Embodiment 4, wherein the clip attachment area further comprises a third electrode in electrical communication with the second conductive pathway.

Embodiment 9. The dilator system of any one of Embodiments 1 through 8, further comprising an inner insulative layer disposed between the first conductive pathway and the dilator body.

Embodiment 10. The dilator system of Embodiment 9, further comprising an outer insulative layer disposed over the first conductive pathway.

Embodiment 11. The dilator system of any one of Embodiments 1 through 10, wherein the first emitter patch is disposed on an anterior side of the distal end and the second emitter patch is disposed on a posterior side of the distal end.

Embodiment 12. The dilator system of any one of Embodiments 1 through 11, wherein actuation of the clip sends an electrical signal to the first emitter patch and subsequently to the second emitter patch.

Embodiment 13. A dilator for use in surgery, the dilator comprising a body extending from a proximal end to a distal end; an attachment area at the proximal end of the body, the attachment area for receiving a stimulating clip, the attachment area comprising an anterior electrode and a posterior electrode, the anterior electrode and posterior electrode insulated from each other; a first conductive pathway extending from the anterior electrode to the distal end of the body; a second conductive pathway extending from the posterior electrode to the distal end of the body; an anterior emitter at a distal end of the first conductive pathway; and a posterior emitter at a distal end of the second conductive pathway, the posterior emitter disposed opposite the anterior emitter.

Embodiment 14. The dilator of Embodiment 13, further comprising a base insulative layer disposed about the body of the dilator, and between the body of the dilator and both the first conductive pathway and the second conductive pathway.

Embodiment 15. The dilator of Embodiment 13 or Embodiment 14, wherein the posterior electrode comprises a first posterior electrode and a second posterior electrode at the attachment area; wherein the anterior electrode is positioned between the first posterior electrode and the second posterior electrode.

Embodiment 16. The dilator of Embodiment 15, wherein the attachment area further comprises an electrode insulative portion disposed between the first posterior electrode and the second posterior electrode.

Embodiment 17. The dilator of any one of Embodiments 13 through 16, further comprising a first insulative layer disposed over the first conductive pathway and a second insulative layer disposed over the second conductive pathway.

Embodiment 18. The dilator of any one of Embodiments 15 through 17, wherein the attachment area at the proximal end of the body comprises a single attachment area containing the anterior electrode, the first posterior electrode, and the second posterior electrode.

Embodiment 19. A method of manufacturing a dilator, the method comprising selecting a body of the dilator, the body extending from a proximal end to a distal end; disposing a base insulative layer about the body; creating a clip attachment portion having a plurality of electrodes near the proximal end of the body; creating a first conductive pathway, the first conductive pathway extending from the clip attachment portion to a first emitter patch at the distal end of the body; creating a second conductive pathway, the second conductive pathway opposite from the first conductive pathway and extending from the clip attachment portion to a second emitter patch at the distal end of the body; and insulating the first conductive pathway.

Embodiment 20. The method of Embodiment 19, wherein creating the clip attachment portion having the plurality of electrodes near the proximal end comprises applying a first conductive layer over the base insulative layer at the clip attachment portion, the first conductive layer extending partially around a circumference of the clip attachment portion; applying a second conductive layer over the base insulative layer at the clip attachment portion, the second conductive layer positioned adjacent to the first conductive layer thereby forming a second electrode, the second electrode in electrical communication with the second conductive pathway; insulating a portion of the second conductive layer; applying a first conductive patch on the insulated portion of the second conductive layer, thereby forming a first electrode, the first electrode in electrical communication with the first conductive pathway.

Embodiment 21. The method of Embodiment 19, wherein creating the clip attachment portion having the plurality of electrodes near the proximal end comprises applying a first conductive layer over the base insulative layer at the clip attachment portion, the first conductive layer extending partially around a circumference of the clip attachment portion; applying a second conductive layer over the base insulative layer at the clip attachment portion, the second conductive layer including a top portion and a bottom portion, the top and bottom portions flanking the first conductive layer; insulating a portion of the first conductive layer; insulating a portion of the second conductive layer; applying a first conductive patch on an insulated portion of the second conductive layer, thereby forming a first electrode, the first electrode in electrical communication with the first conductive pathway; and applying a second conductive patch on an insulated portion of the first conductive layer, thereby forming a second electrode, the second electrode in electrical communication with the second conductive pathway.

Embodiment 22. The method of Embodiment 19 or Embodiment 21, wherein creating the first conductive pathway comprises applying a first conductive ink over the base insulative layer at the clip attachment portion to form a first electrode; extending the first conductive ink from the clip attachment portion to the distal end; forming the first emitter patch at the distal end of the body; insulating a portion of the first conductive ink; and applying a first conductive patch on an insulated portion of the second conductive pathway, the first conductive patch in electrical communication with the first electrode and the first emitter patch.

Embodiment 23. The method of Embodiment 22, wherein applying the first conductive ink over the base insulative layer comprises printing and/or etching conductive ink onto the base insulative layer.

Embodiment 24. The method of Embodiment 22 or Embodiment 23, wherein applying the first conductive patch comprises printing the first conductive ink on top of the insulated portion of the second conductive pathway, such that the first conductive ink is electrically insulated from the second conductive pathway; and connecting the first conductive patch to the first electrode.

Embodiment 25. The method of any one of Embodiments 19 through 24, wherein creating the second conductive pathway comprises applying a second conductive ink over the base insulative layer at the clip attachment portion to form a second electrode; extending the second conductive ink from the clip attachment portion to the distal end; forming the second emitter patch at the distal end of the body; insulating a portion of the second conductive ink; and applying a second conductive patch on an insulated portion of the first conductive pathway, the second conductive patch in electrical communication with the second electrode and the second emitter patch.

Embodiment 26. An intraoperative monitoring system comprising a dilator having a clip attachment area near a proximal end of the dilator, the clip attachment area being electrically conductive; a clip receivable by the clip attachment area, the clip for conducting an electrical signal to first and second emitter patches disposed at a distal end of the dilator; and a neuromonitoring system in communication with the clip, the neuromonitoring system selectively conducting the electrical signal to the first emitter patch and the second emitter patch.

Embodiment 27. The intraoperative monitoring system of Embodiment 26, wherein the first and second emitter patches are disposed on opposite sides of the distal end of the dilator.

Embodiment 28. The intraoperative monitoring system of Embodiment 26 or Embodiment 27, wherein the clip is receivable by the clip attachment area in any orientation.

Embodiment 29. The intraoperative monitoring system of any one of Embodiments 26 through 28, wherein the clip attachment area is configured to allow the clip to be attached at one of two positions and to be moved between the two positions during operation of the intraoperative monitoring system.

Embodiment 30. The intraoperative monitoring system of any one of Embodiments 26 through 29, wherein the clip comprises an actuator and the neuromonitoring system conducts the electrical signal upon actuation of the actuator.

Embodiment 31. The intraoperative monitoring system of any one of Embodiments 26 through 30, wherein the neuromonitoring system conducts the electrical signal to the first emitter patch asynchronously to the second emitter patch.

Embodiment 32. A method of using a dilator system, the method comprising attaching a clip to an attachment area of a dilator, the attachment area being electrically conductive; actuating an actuator of the clip to generate an electrical signal; conducting the electrical signal from the attachment area to a first emitter patch disposed at a distal end of the dilator; and conducting the electrical signal from the attachment area to a second emitter patch disposed at the distal end of the dilator, the second emitter patch disposed opposite the first emitter patch, wherein conducting the electrical signal to the first emitter patch or the second emitter patch does not require rotation of the dilator.

Embodiment 33. The method of Embodiment 32, wherein conducting the electrical signal to the first emitter patch is not simultaneous to conducting the electrical signal to the second emitter patch.

Embodiment 34. The method of Embodiment 32 or Embodiment 33, further comprising selecting which of the first emitter patch and the second emitter patch to conduct the electrical signal to.

Embodiment 35. The method of Embodiment 34, wherein a neuromonitoring system in communication with the clip selects which of the first emitter patch and the second emitter patch the electrical signal is conducted to.

Embodiment 36. A method of using a dilator system, the method comprising attaching a clip to an attachment area of a dilator, the attachment area being electrically conductive and the clip in communication with a neuromonitoring system; actuating an actuator of the clip to generate an electrical signal; conducting, by the neuromonitoring system, the electrical signal from the attachment area to a first emitter patch disposed at a distal end of the dilator; and conducting, by the neuromonitoring system, the electrical signal from the attachment area to a second emitter patch disposed at the distal end of the dilator, the second emitter patch disposed opposite the first emitter patch, wherein conducting the electrical signal to the first emitter patch or the second emitter patch does not require rotation of the dilator.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular surgical procedure (e.g., a spinal procedure); however, other procedures where use of a dilator is helpful or necessary are also contemplated. Structures that are closer to a clinician or surgeon are referred to as more “proximal” while structures that extend away from the clinician or surgeon are referred to as “distal.”

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A dilator system for use in surgery, the dilator system comprising:
a dilator body extending from a proximal end to a distal end;
a clip attachment area near the proximal end of the dilator body, the clip attachment area being electrically conductive;
a first emitter patch at the distal end;
a first conductive pathway extending from the clip attachment area to the first emitter patch to electrically connect the clip attachment area to the first emitter patch;
a second emitter patch at the distal end, the second emitter patch opposing the first emitter patch;
a second conductive pathway extending from the clip attachment area to the second emitter patch to electrically connect the clip attachment area to the second emitter patch; and
a clip receivable by the clip attachment area, the clip for conducting an electrical signal to the first emitter patch or the second emitter patch.

2. The dilator system of claim 1, wherein the first emitter patch and the first conductive pathway comprise a conductive ink.

3. The dilator system of claim 1, wherein the clip attachment area comprises a first electrode in electrical communication with the first conductive pathway and a second electrode in electrical communication with the second conductive pathway.

4. The dilator system of claim 3, wherein the first electrode and the second electrode are electrically insulated from each other at the clip attachment area.

5. The dilator system of claim 3, wherein the first electrode is disposed distally relative to the second electrode.

6. The dilator system of claim 3, wherein the clip comprises a first pin for alternating electrical connection to the first electrode and the second electrode, which alternating connection is achieved by moving the clip from a first position on the dilator to a second position on the dilator and then back to the first position.

7. The dilator system of claim 3, wherein the clip comprises a first pin for electrical connection to the first electrode and a second pin for electrical connection to the second electrode.

8. The dilator system of claim 3, wherein the clip attachment area further comprises a third electrode in electrical communication with the second conductive pathway.

9. The dilator system of claim 1, further comprising an inner insulative layer disposed between the first conductive pathway and the dilator body.

10. The dilator system of claim 9, further comprising an outer insulative layer disposed over the first conductive pathway.

11. The dilator system of claim 1, wherein the first emitter patch is disposed on an anterior side of the distal end and the second emitter patch is disposed on a posterior side of the distal end.

12. A dilator for use in surgery, the dilator comprising:
a body extending from a proximal end to a distal end;
an attachment area at the proximal end of the body, the attachment area for receiving a stimulating clip, the attachment area comprising an anterior electrode and a posterior electrode, the anterior electrode and posterior electrode insulated from each other;
a first conductive pathway extending from the anterior electrode to the distal end of the body;
a second conductive pathway extending from the posterior electrode to the distal end of the body;
an anterior emitter at a distal end of the first conductive pathway; and
a posterior emitter at a distal end of the second conductive pathway, the posterior emitter disposed opposite the anterior emitter.

13. The dilator of claim 12, further comprising a base insulative layer disposed about the body of the dilator, and between the body of the dilator and both the first conductive pathway and the second conductive pathway.

14. The dilator of claim 12, wherein the anterior electrode is positioned either distally or proximally of the posterior electrode.

15. The dilator of claim 12, wherein the posterior electrode comprises a first posterior electrode and a second posterior electrode at the attachment area; and wherein the anterior electrode is positioned between the first posterior electrode and the second posterior electrode.

16. The dilator of claim 15, wherein the attachment area further comprises an electrode insulative portion disposed between the first posterior electrode and the second posterior electrode.

17. The dilator of claim 15, further comprising a first insulative layer disposed over the first conductive pathway and a second insulative layer disposed over the second conductive pathway.

18. The dilator of claim 17, wherein the attachment area at the proximal end of the body comprises a single attachment area containing the anterior electrode, the first posterior electrode, and the second posterior electrode.

19. A method of manufacturing a dilator, the method comprising:
selecting a body of the dilator, the body extending from a proximal end to a distal end;
disposing a base insulative layer about the body;
creating a clip attachment portion having a plurality of electrodes near the proximal end of the body;
creating a first conductive pathway, the first conductive pathway extending from the clip attachment portion to a first emitter patch at the distal end of the body;
creating a second conductive pathway, the second conductive pathway opposite from the first conductive pathway and extending from the clip attachment portion to a second emitter patch at the distal end of the body; and
insulating the first conductive pathway.

20. The method of claim 19, wherein creating the clip attachment portion having the plurality of electrodes near the proximal end comprises:
applying a first conductive layer over the base insulative layer at the clip attachment portion, the first conductive layer extending partially around a circumference of the clip attachment portion;
applying a second conductive layer over the base insulative layer at the clip attachment portion, the second conductive layer including a top portion and a bottom portion, the top portion and bottom portion flanking the first conductive layer;
insulating a portion of the first conductive layer;
insulating a portion of the second conductive layer;
applying a first conductive patch on an insulated portion of the second conductive layer, thereby forming a first electrode, the first electrode in electrical communication with the first conductive pathway; and
applying a second conductive patch on an insulated portion of the first conductive layer, thereby forming a second electrode, the second electrode in electrical communication with the second conductive pathway.
